(19) United States
(12) Patent Application Publication
(10) Pub. No.: US 2015/0361478 A1
(43) Pub. Date: Dec. 17, 2015

(54) ANIMAL FEED ENZYME EXTRACTION

(71) Applicant: BASF ENZYMES LLC, San Diego, CA (US)

(72) Inventors: Cristina POP, Solana Beach, CA (US); Arne I. SOLBAK, JR., San Diego, CA (US); Adrienne HUSTON DAVENPORT, San Diego, CA (US)

(21) Appl. No.: 14/764,142

(22) PCT Filed: Jan. 28, 2014
(86) PCT No.: PCT/US14/13282

§ 371 Date: Jul. 28, 2015

Related U.S. Application Data
(60) Provisional application No. 61/758,070, filed on Jan. 29, 2013.

(30) Foreign Application Priority Data
Mar. 5, 2013 (GB) 1303876.5

(51) Int. Cl. C12Q 1/42

(52) U.S. Cl. CPC C12Q 1/42; G01N 2333/916

(57) ABSTRACT

An in-feed assay and buffers for extracting an enzyme from a feed pellet treated at high temperature is provided. Another embodiment of the invention is measuring the enzyme activity of the enzyme additive extracted from the animal feed, measuring the quantity of enzyme extracted from the animal feed, or measuring both. In one embodiment the enzyme is an animal feed additive, such as a phytase.

### FIELD OF THE INVENTION

The embodiments disclosed herein relate to compositions and methods for the quantification of enzyme activity in feed and nutrient pellets.

### BACKGROUND OF THE INVENTION

Bioavailable carrier products such as animal feed pellets, plant fertilizer pellets, or pellet products for pest control, are often fortified with one or more enzymes to increase the bioavailability of their components. This fortification can have an effect on the utility of the pellet, increasing the effectiveness of the pellet, decreasing the amount of pellet product needed to satisfy customer demand, and decreasing waste products resulting from pellet use.

To produce a product having a consistent performance, it is important that the amount of enzymatic activity in each batch be accurately measurable. However, the pelletization process often involves heat treatments of as high as 70° C., 75° C., 80° C., 85° C., 88° C., 90° C., 93° C., 95° C., or higher temperatures. Higher temperatures are often used, as they may reduce processing time, produce pellets less likely to crumble and, ultimately, reduce product cost.

However, these heat treatments can complicate quantification of enzymatic activity in a number of ways. For example, heat treatment may inactivate enzymes in a pellet, or make it substantially more difficult to extract enzymes without destroying their activity, or both. As a result, producing pellet products with a consistent, known enzyme activity is a major challenge.

Phytase is an example of an enzyme that can have an effect as a supplement in animal feed pellets. Phytase degrades phytic acid into a myo-inositol core and one or more free phosphate molecules. Phytic acid consists of a myo-inositol core to which are covalently attached six phosphate groups. Phytic acid is a constituent of the plant material, such as soy bean seeds, that are used to generate feed pellets for animals such as non-ruminant animals, e.g. poultry, broilers, birds, chickens, layers, turkeys, ducks, geese, and fowl; ruminant animals e.g. cows, cattle, horses, and sheep; pigs, swine, piglets, growing pigs, and sows; companion animals including but not limited to: cats, dogs, rodents, and rabbits; fish including but not limited to salmon, trout, tilapia, catfish and carp; and crustaceans including but not limited to shrimp and prawn. Because these animals are unable to fully digest phytic acid, phytic acid has a number of detrimental effects. It chelates divalent cations such as Calcium and Magnesium, and its phosphate is in a form that is biologically unavailable to the animals being fed, resulting in a need to supplement the animal diet with these nutrients despite their being abundant in the feedstock. Furthermore, because these nutrients pass through the animal undigested, they are available to decomposers further down the food chain that are able to degrade phytic acid, resulting in, for example, algal blooms in surface waters to which the animal effluent comes into contact.

For example, it has been shown that phytase addition to corn-soybean diets may improve apparent total tract digestibility of phosphate in finishing pigs (Kerr et al., (2012) “Effect of phytase on apparent total tract digestibility of phosphorous in corn-soybean meal diets fed to finishing pigs,” J. Animal Sci. 88: 238-247), young chickens (Pirgozliev (2011) “The effects of supplementary bacterial phytase on dietary energy and total tract amino acid digestibility when fed to young chickens,” Br. Poult. Sci. 52(2):245-54) and broiler chickens (Powell (2011) “Phytase supplementation improved growth performance and bone characteristics in broilers fed varying levels of dietary calcium,” Poult Sci. 90(3):604-8; Woyengo (2011) “Growth performance and nutrient utilization of broiler chickens fed diets supplemented with phytase alone or in combination with citric acid and multicarbohydrase,” Poult Sci. 89(10):2221-9).

Currently, the standard for measuring phytase activity in an animal feed is by using the Association of Analytical Communities (AOAC) method number 2000.12. This method yields inconsistent results for measuring phytase activity that is extracted from the animal feed when the feed is subjected to high temperatures used in the animal feed pelleting process. A problem with this method is that when phytase is extracted from the animal feed, the level of phytase activity may be lower than the level of phytase amount that is actually present in the animal feed. In other words, the current methods for extracting phytase cannot extract 100% of the phytase activity from the animal feed, when the feed is prepared at high temperature.

However, improving measurement of phytase activity in animal feed has long been a challenge. Kim et al., (2005) “An improved method for a rapid determination of phytase activity in animal feed” J. Anim Sci. 2005, 83:1062-1067, developed improved methods for reducing phosphate background in measurements using an AOAC-based method. Basu et al, (U.S. Pat. No. 7,629,139 B2) using a Sodium Borate based buffer, were able to isolate 68%, 23% and 13% of the initial phytase activity in pellets heat treated at 70° C., 83° C., and 86° C.

Furthermore, a component of adding enzymes, such as a phytase to an animal feed, is that one skilled in the art will need to know how much phytase activity is present in the animal feed. If the current AOAC methods for extracting phytase from an animal feed are only extracting about 60% to 70% of the phytase from a feed pellet treated at 75 degrees C. and less than 15% of the phytase at 86 degrees C., then it is difficult to measure the total amount of phytase activity because there is only a fraction of the total active phytase to measure. As has been noted by researchers using the AOAC method, “the results provided by the analytical method are expressed as an activity U/kg and not on a mass basis (mg/kg),” Grizzi et al., (2008) “Determination of Phytase Activity in Feed: Interlaboratory Study” Journal of AOAC International 91(2):259, at 261.

Therefore a need exists to provide a method for extracting more than 70% of the phytase from an animal feed for animal feed pellets generated at high temperatures.

More generally, an unmet need exists for the development of methods and compositions for the accurate quantification of enzymatic activity in heat treated pellet products.

### SUMMARY OF THE INVENTION

Some embodiments disclosed herein comprise an aqueous composition for extraction of polypeptides from heat-treated solids, comprising a bile-salt detergent, a denaturant, a base, and water.

In some aspects the denaturant is a chaotropic agent. In some aspects the denaturant is selected from the list consisting of Urea, a guanidinium salt, and a perchlorate salt. In some aspects the denaturant is urea. In some aspects the urea is present in the composition at a concentration of from about 0.0 M to about 3.0 M. In some aspects the urea is present in the composition at a concentration of about 1 M.

In some aspects the base is a bicarbonate salt. In some aspects the sodium bicarbonate is present at a concentration of about 50 mM to about 200 mM. In some aspects the sodium bicarbonate is present at a concentration of about 100 mM.

In some aspects the aqueous composition has a basic pH. In some aspects the aqueous composition has a pH of about 8.0 to about 11.0. In some aspects the aqueous composition has a pH of about 8.5 to about 10.5. In some aspects the aqueous composition has a pH of about 10. In some aspects the aqueous composition has a pH of 10.

In some aspects the bile-salt detergent comprises a steroid acid selected from the list consisting of taurocholic acid, glycocholic acid, cholic acid, deoxycholic acid, lithocholic acid, chenodeoxycholic acid, and any combination thereof. In some aspects the bile-salt detergent comprises a sodium cation. In some aspects the bile-salt detergent is sodium deoxycholate. In some aspects the sodium deoxycholate is present at a concentration from about 0.25% to about 3.0%. In some aspects the sodium deoxycholate is present at a concentration of 1%.

Some embodiments comprise an aqueous composition comprising 100 mM sodium bicarbonate pH10.0, 1.0% sodium deoxycholate and 1 M urea.

Some embodiments comprise a method of extracting polypeptides from a heat-treated feed pellet, comprising the steps of: providing a heat-treated feed pellet comprising polypeptides, wherein the pellet has been heat-treated to at least 70° C., contacting the heat treated feed pellet with an aqueous solution, agitating the heat-treated feed pellet in contact with the aqueous solution, and separating the polypeptide from the heat treated feed pellet into the aqueous solution. In some aspects the method further comprises creating an environment in the aqueous solution having detergents at a level about or greater than a critical micelle concentration, and contacting the heat treated feed pellet with a mild denaturant, wherein the mild denaturant disrupts intermolecular hydrophobic protein—protein interactions. In some aspects the method further comprises providing an aqueous solution comprising detergents at a level about or greater than a critical micelle concentration. In some embodiments the method further comprises providing a mild denaturant, wherein said mild denaturant disrupts intermolecular hydrophobic protein—protein interactions.

In some aspects the agitation comprises containing the heat treated solid with the aqueous solution and vortexing the contained heat treated solid and the aqueous solution. In some aspects the method comprising incubating the heat treated solid in contact with the aqueous solution.

In some aspects the method comprises incubation and gentle agitation. In some aspects the incubation comprises a temperature of about 20° C. to 40° C., or up to the melting temperature of the polypeptide. In some aspects the incubation comprises a temperature of about 20° C. to 40° C., or up to the denaturation temperature of the polypeptide

In some aspects the polypeptides are enzymes. In some aspects the enzymes retain catalytic activity following subjection to the method.

In some aspects the heat-treated solid is subject to a heat treatment of at least 75° C. prior to the contacting. In another aspect, the heat-treated solid is an animal feed composition. In another aspect, the animal feed composition is a solid and/or semi-solid form that has been subjected to a heat-treatment step, wherein the animal feed composition is a pellet, a tablet, a pill, a gel, a granule, a coated granule, a ground grain, a powder, or any combination thereof. In another aspect the heat-treated solid is an animal feed pellet. In a further aspect, the heat-treated solid or animal feed pellet is contacted with an aqueous composition for the extraction of polypeptides from the heat-treated solid or animal feed pellet wherein the aqueous composition comprises a bile-salt detergent, selected from taurocholic acid, glycocholic acid, cholic acid, deoxycholic acid, lithocholic acid, chenodeoxycholic acid, and any combination thereof; a denaturant selected from Urea, a guanidinium salt, and a perchlorate salt; a bicarbonate salt; and water.

Some embodiments comprise a low stringency buffer comprising: about 50 mM Tris pH 8.0, about 0.01% Tween20, and about 10 mM CaCl₂. In some aspects the low stringency buffer comprises: 50 mM Tris pH 8.0, 0.01% Tween20, and 10 mM CaCl₂.

Some embodiments comprise a high stringency buffer selected from the list consisting of: a) a Urea buffer comprising about 8.0 M Urea, 50 mM Tris pH 8.0; b) a Guanidine buffer comprising about 6.0 M Guanidine, 50 mM Tris pH 8.0; and c) a mRIPA buffer comprising about 50 mM Tris pH 7.6, 0.15 M NaCl, 0.1% SDS, 0.5% sodium-deoxycholate, and 1% Triton-100. Some embodiments comprise a high stringency buffer selected from the list consisting of: a) a Urea buffer comprising 8.0 M Urea, 50 mM Tris pH 8.0; b) a Guanidine buffer comprising 6.0 M Guanidine, 50 mM Tris pH 8.0; and c) a mRIPA buffer comprising 50 mM Tris pH 7.6, 0.15 M NaCl, 0.1% SDS, 0.5% sodium-deoxycholate, and 1% Triton X-100.

Some embodiments comprise a method for measuring the amount of enzyme in a heat-treated pellet comprising: (a) providing a mash comprising an enzyme additive wherein the enzyme activity is known; (b) providing a heat treated pellet generated from the mash; (c) treating the mash and the heat treated pellet with a low stringency buffer and measuring the enzyme activity extracted from the mash and the pellet; (d) treating the mash and the heat treated pellet with a high stringency buffer and measuring the total enzyme amount extracted from the mash and the pellet; and, (e) determining an enzyme activity of the enzyme extracted from the heat treated pellet.

In some aspects the low stringency buffer comprises water, about 50 mM Tris pH8.0, about 0.01% Tween20, and about 10 mM CaCl₂.

In some aspects the low stringency buffer comprises water, 50 mM Tris pH8.0, 0.01% Tween20, and 10 mM CaCl₂.

In some aspects the high stringency buffer is selected from the list consisting of: a) a Urea buffer comprising about 8.0 M Urea, 50 mM Tris pH 8.0; b) a Guanidine buffer comprising about 6.0 M Guanidine, 50 mM Tris pH 8.0; and c) a mRIPA buffer comprising about 50 mM Tris pH 7.6, 0.15 M NaCl, 0.1% SDS, 0.5% sodium-deoxycholate, and 1% Triton X-100. In some embodiments the high stringency buffer is selected from the list consisting of: a) a Urea buffer comprising 8.0 M Urea, 50 mM Tris pH 8.0; b) a Guanidine buffer comprising 6.0 M Guanidine, 50 mM Tris pH 8.0; and c) a mRIPA buffer comprising 50 mM Tris pH 7.6, 0.15 M NaCl, 0.1% SDS, 0.5% sodium-deoxycholate, and 1% Triton X-100. In some aspects the high stringency buffer is a composition comprising 100 mM sodium bicarbonate pH10.0, 1.0% sodium deoxycholate and 1M urea.

In some aspects the enzyme is an animal feed enzyme additive. In some aspects the enzyme is selected from a listing consisting of: a phytase, cellulase, lactase, lipase, protease, catalase, xylanase, beta-glucanase, mannanase, amylase, amidase, epoxide hydrolase, esterase, phospholipase, transaminase, amine oxidase, cellobiohydrolase, ammonia lyase, or any combination thereof.

In some aspects the quantity of enzyme is determined by ELISA.

### BRIEF DESCRIPTION OF THE DRAWINGS

FIG. 1 depicts the percent enzyme activity extracted from a feed pellet using a Single Buffer (II) and the Modified AOAC Buffer, relative to the amount enzyme extracted from a similar amount of feed mash not subject to heat treatment.

FIG. 2 depicts the percent of phytase (mass) extracted from an animal feed pellet treated at high temperatures (heat-treated solid) using high stringency Buffer I, relative to the amount of enzyme extracted from a similar amount of mash or pre-pelleted mash not subject to heat treatment.

### DETAILED DESCRIPTION

Aspects of the invention relate to pelletized animal feed or nutrient products. For simplicity, the disclosure herein will focus on the example of animal feed pellets. However, embodiments disclosed herein may demonstrate equal utility when applied to a number of delivery matrices such as a pellet, a tablet, a gel, a liquid, a spray, a ground grain, a powder, or any combination thereof.

Some embodiments disclosed herein comprise methods for extracting an enzyme additive from a pelletized feed, an extruded feed, an animal feed, a food, a cereal bar, or a nutrient product. Some embodiments disclosed herein comprise methods for extracting an enzyme additive from the product which substantially preserves the enzyme activity. Some embodiments disclosed herein comprise measuring the enzyme activity of the enzyme additive extracted from the product, measuring the quantity of enzyme extracted from the product, or measuring both. In some embodiments the product is an animal feed directed to a monogastric animal and the enzyme is a phytase.

Animal feed is produced from a variety of ingredients comprising various combinations of plants, animals, edible by-products, and additives, such as vitamins, minerals, enzymes, and other nutrients (SAPKOTA; Environ Health Perspect. 2007 May; 115(5): 663-670).

Animal feed additives, such as enzymes, are designed, for example, to decrease gut viscosity and/or increase the nutritional value of the feed by releasing nutrients and allowing increased absorption of essential vitamins and minerals in the animal, which in turn, enhances animal product yield, while reducing harmful materials in animal waste.

Animal feed enzyme additives include, but are not limited to: a phytase, cellulase, lactase, lipase, protease, catalase, xylanase, beta-glucanase, mannanase, amylase, amidase, epoxide hydrolase, esterase, phospholipase, transaminase, amine oxidase, cellobiohydrolase, ammonia lyase, or any combination thereof. In some embodiments, any enzyme or combination of enzymes used as additives for a feed can be extracted using the compositions or methods of the invention.

In some embodiments, a phytase is a phosphoric monoester hydrolase enzyme that catalyzes hydrolysis of phytic acid (myo-inositol-hexakisphosphate) to phosphate and myo-inositol having fewer than six phosphate groups. According to the recommendations of the Nomenclature Committee of the International Union of Biochemistry and Molecular Biology (IUBMB) and Bairoch A., “The ENZYME database in 2000,” Nucleic Acids Res 28:304-305(2000), a phytase may be described in a variety of names and identifying numbers. In another embodiment, a phytase is characterized as having Enzyme Commission (EC) number EC 3.1.3.8, and are also referred to as: 1-phytase; myo-inositol-hexakisphosphate 3-phosphohydrolase; phytate 1-phosphatase; phytate 3-phosphatase; or phytate 6-phosphatase. In another embodiment a phytase is characterized as EC 3.1.3.26, also referred to as: 4-phytase; 6-phytase (name based on 1L-numbering system and not 1D-numbering); or phytate 6-phosphatase. In another embodiment a phytase is characterized as EC 3.1.3.72, also referred to as 5-phytase. In another embodiment a phytase is a histidine acid phosphatase (HAP); a β-propeller phytase; purple acid phosphatase (PAP); and protein tyrosine phosphatase (PTPs). In some embodiments, a phytase is described using nomenclature know in the art.

An animal feed comprising a phytase of the invention can be provided in any animal feed formulation known to those skilled in the art. Examples of an animal feed formulation include, but are not limited to a feed treated at high temperature, wherein the formulation is selected from a group consisting of: a delivery matrix, a pellet, a tablet, a gel, a liquid, a granule, a spray, a ground grain, a powder, or any combination thereof.

An embodiment of this invention comprises extracting a phytase from an animal feed, measuring the amount of phytase extracted, and measuring the amount of phytase activity of the phytase extracted from the animal feed. There are many examples of a phytase that can be used as an additive for animal feed, including but not limited to: PHYZYME (Dupont, Danisco, Genencor); QUANTUM and FINASE (AB Vista, AB Enzymes); NATUPHOS (BASF); RONOZYME (DSM); BIOFEED phytase (Novo Nordisk); ALLZYME phytase (Alltech); OPTIPHOS (Enzyvia, Phytex, Cornell); and ROVABIO (Adisseo). In some embodiments, the phytase activity to be assayed is a thermostable enzyme activity.

Some embodiments of this invention are methods for extracting an enzyme from an animal feed and measuring the activity, measuring the quantity, or both of the enzyme used as additive for animal feed.

Some embodiments of this invention are methods for extracting an enzyme, such as a phytase, from an animal feed, wherein at least 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the enzyme or of the enzymatic activity, such as a phytase or phytase activity, is extracted from the animal feed.

In some embodiments an enzyme, such as phytase, is extracted from a pelletized animal or plant product such as an animal feed following a pelleting process involving heat treatment. In some embodiments, the pelleting process comprises treatment at a temperature of at least 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., or above 69° C. In some embodiments, the pelleting process comprises treatment at a temperature of 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or more than 99° C. In some embodiments the extracted enzyme may be used to determine the total enzymatic activity in the pelletized animal or plant product.

In some embodiments an enzyme, such as phytase, retains as least: 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the enzyme activity when compared to the enzyme added to the mash prior to the animal feed pelleting process.

In some embodiments an enzyme, such as phytase, is extracted from an animal feed using a buffer. In some embodiments the buffer is the AOAC buffer comprising distilled water plus 0.01% Tween 20 (GIZZI, J. of AOAC International, Vol. 91, No. 2, 2008)

Some embodiments of this invention provide a modified AOAC buffer, also known herein as a low stringency buffer. The modified AOAC extraction uses a composition comprising: 50 mM Tris pH 8.0, 0.01% Tween 20, 10 mM CaCl₂. Low stringency buffers may produce low yields of a phytase activity when the phytase is extracted from an animal feed pelleted at high temperature. In some embodiments, when a low stringency buffer is used not all of the phytase is extracted from an animal feed, but the phytase recovered is likely to retain its activity. In some embodiments 20%, 25%, 30%, 35%, 40%, 45%, 50%, 55%, 60%, 65%, 70%, 75%, 80%, 85%, 90%, 95% or more than 95% of activity is retained. As a result, a level of phytase activity can be measured accurately. However, when the low stringency buffers are used to extract enzyme activities from pellets produced through a heat treatment of above 75° C., such as at temperatures up to 93° C., the level of enzyme activity such as phytase activity recovered decreases to less than 10%.

A need exists to provide a method for extracting more phytase from an animal feed following a high temperature pelleting process.

Some embodiments provide modifications to the AOAC method, wherein the modifications enable an improved determination of enzyme activity such as phytase activity in pellets generated through a high-temperature pelletization method. An aspect of this embodiment is a High Stringency Buffer (Buffer 1). In some embodiments a high stringency buffer is Buffer 1(a), wherein high stringency buffer 1(a) is a composition comprising: Urea buffer: 8.0 M Urea, 50 mM Tris pH 8.0. In some embodiments a high stringency buffer is Buffer 1(b), wherein buffer 1(b) is a composition comprising: Guanidine buffer: 6.0 M Guanidine, 50 mM Tris pH 8.0. In some embodiments a high stringency buffer is Buffer 1(c), wherein Buffer 1(c) is a composition comprising: RIPA buffer: 50 mM Tris pH 7.6+0.15 M NaCl+0.1% SDS+0.5% sodium-deoxycholate+1% Triton X100.

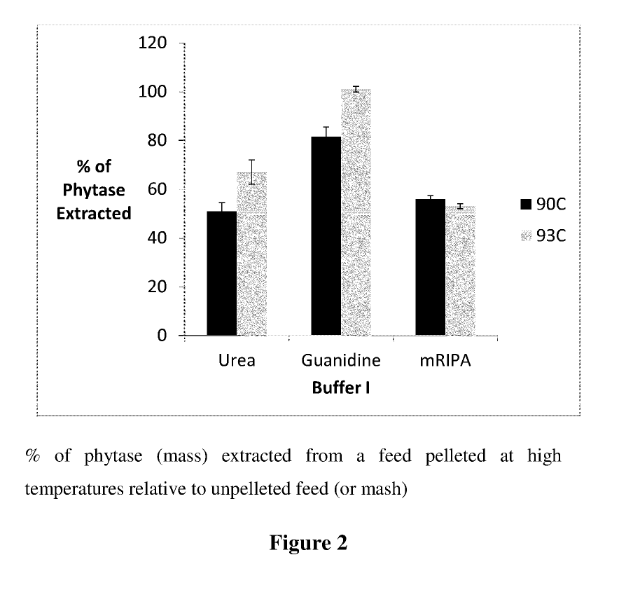

The High Stringency Buffer can extract at least 47% to 100% of the total phytase amount from a pelletized, high-temperature treated animal feed relative to untreated feed precursor such as mash. The high stringency buffers extract phytase from animal feed pellets produced at high temperatures at much higher percent yields than other buffers. See Table 1, or FIG. 2. However, when the phytase is extracted using a high stringency buffer, the phytase activity of that phytase is reduced, or in some embodiments completely destroyed (not shown).

In some embodiments a combination of a low stringency buffer and a high stringency buffer is used to assay both the total amount of an enzyme such as phytase present in a high-temperature treated pellet using a high-stringency buffer; and to assay the activity per unit of protein extracted by using a low stringency buffer to extract enzyme activity such as phytase activity from a high-temperature treated pellet, and to quantify the amount of enzyme present in the low-stringency extract, for example by using means known in the art such as an ELISA assay.

### Determining Total Enzymatic Activity in High-Temperature Treated Pellets Using a Low and High Stringency Buffer in Combination

In some embodiments a low stringency buffer and a high stringency buffer are used in combination to determine the enzymatic activity, such as phytase activity, in a pellet such as an animal feed pellet. For example, a low stringency buffer such as that disclosed herein may be used to extract a phytase enzyme from a pellet under conditions such that the extraction process does not harm the overall activity of the extracted enzyme. Following extraction with a low stringency buffer, the extract may be assayed for both enzymatic activity and total amount of protein extracted. Enzyme activity and the total amount of protein extracted may be determined using methods available to one of skill in the art.

From the above, one of skill in the art may obtain the enzymatic activity per unit protein in the extract, which will correspond to the enzyme activity per unit of protein in the heat-treated pellet.

However, low stringency buffers disclosed herein may extract substantially less than the total protein content of a pellet, particularly if the pellet has been subject to a high-temperature heat pretreatment.

Subjecting a pellet to a high stringency buffer such as those disclosed herein may allow one of skill in the art to extract all or substantially all of an enzyme contained within a heat-treated pellet. However, this extraction is likely to negatively impact enzymatic activity of the extracted protein.

As a further aspect of some embodiments disclosed herein, the total enzymatic activity in a pellet of a given batch is determined by the use of both a low stringency and a high stringency buffer in combination. The low stringency buffer may be used as disclosed above to determine the activity per unit protein in a heat-treated pellet or pellet batch. The high stringency buffer may be used to determine the total amount of protein in an average pellet of a given batch. By multiplying the activity per unit protein, determined using the low stringency buffer, with the total protein content per pellet, determined using the high stringency buffer, one may calculate the total enzymatic activity in a heat treated pellet or pellet batch.

### Determining Total Enzymatic Activity in High-Temperature Treated Pellets Using a Single Buffer System

In some embodiments a single buffer may be used to extract all of the enzyme, wherein the enzyme retains all of the enzyme activity when the enzyme is extracted from a high-temperature pelletized product such as an animal feed when compared to the enzyme added to the mash prior to the high-temperature pelleting process.

In some embodiments, the enzyme is a phytase, wherein the phytase activity is not destroyed following a heat treatment and an extraction using a single buffer. In another embodiment following a heat treatment and an extraction using a single buffer, the phytase activity can be measured and compared to the phytase activity of the phytase added prior to the heat treatment.

In some embodiments, the enzyme is a Xylanase, wherein the xylanase activity is not destroyed following a heat treatment and an extraction using a single buffer. In another embodiment following a heat treatment and an extraction using a single buffer, the xylanse activity can be measured and compared to the xylanase activity of the xylanase added prior to the heat treatment.

In some embodiments, the enzyme is any animal feed enzyme additive, wherein the animal feed enzyme additive is not destroyed following a heat treatment and an extraction using a single buffer. In another embodiment following a heat treatment and an extraction using a single buffer, the animal feed enzyme additive can be measured and compared the animal feed enzyme additive enzyme activity of the animal feed enzyme additive added prior to the heat treatment.

Some embodiments provide a single buffer comprising a bile-salt detergent, a denaturant, a base, and water. The single buffer will produce high yields of a phytase extracted from an animal feed pelleted at high temperature and all phytase activity is preserved in the phytase extracted from the animal feed.

Without being bound by theory, it is believed that high stringency buffers such as those disclosed above may decrease enzymatic activity of extracted proteins by denaturing the proteins, thus abolishing or decreasing total enzymatic activity or enzymatic activity per protein. This denaturation may result in part from the absence of a hydrophobic environment in the buffers. Buffers stringent enough to release proteins from high temperature treated pellets may also disrupt hydrophobic intramolecular interactions within individual enzymes, leading to a loss of enzymatic activity in extracted proteins.

In some embodiments disclosed herein, protein extraction involves a buffer having a hydrophobic component at a concentration sufficient to maintain an extraction environment at above a critical micelle density, such that the buffer maintains micelles into which extracted proteins may be sequestered such that their hydrophobic interactions may be preserved. In some embodiments, the hydrophobic component comprises a bile-salt detergent. Examples of compounds that form bile-salt detergents include taurocholic acid, glycocholic acid, cholic acid, deoxycholic acid, lithocholic acid, and chenodeoxycholic acid, and any combination thereof. In another embodiment the detergent is selected from CHAPS, tween20, triton X100, and any combination thereof. In some embodiments, the concentration of detergent varies from at least 0.0% up to about 3.0% of the composition. In another embodiment the concentration is about 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, or 3.0%.

In some embodiments a Single Buffer System (Buffer II) comprises about: 100 mM NaHCO3 (sodium bicarbonate); about pH 10.0; about 1.0% Na-deoxycholate; and about 1.0 M Urea. In some embodiments a Single Buffer System (Buffer II) comprises: 100 mM NaHCO3(sodium bicarbonate); pH 10.0; 1.0% Na-deoxycholate; and 1.0 M Urea. Buffers such as that disclosed above allow the extraction of a high proportion of an enzyme from an animal feed subject to a high temperature pelletization process, without also denaturing said protein such that it loses enzymatic activity. In some embodiments, the single buffer (buffer II) extracts at least 23% to 45% more phytase from an animal feed pelleted at a high temperature, when compared to the amount of phytase added to the mash prior to the pelleting process. See Table 2.

In some embodiments the buffer comprises NaCl, a Na-Borate, a CAPS, or a NaHCO3. In some embodiments the concentration of a buffer constituent ranges from 50 mM to 200 mM. In another embodiment the concentration is 50 mM, 55 mM, 60 mM, 65 mM, 70 mM, 75 mM, 80 mM, 85 mM, 90 mM, 95 mM, 100 mM, 105 mM, 110 mM, 120 mM, 125 mM, 130 mM, 135 mM, 140 mM, 145 mM, 150 mM, 155 mM, 160 mM, 165 mM, 170 mM, 175 mM, 180 mM, 185 mM, 190 mM, 195 mM, 200 mM,or more than 200 mM.

In some embodiments, the pH is in a range from about pH 8.0 to about pH 11.0. In another embodiment the pH is about pH 8.0, pH 8.5, pH 9.0, pH 9.5, pH 10.0, pH 10.5, or pH 11.0.

In some embodiments, the concentration of Urea is in a range from at least 0.0M to 3.0M. In another embodiment, the concentration of Urea is 0.0M, 0.1M, 0.2M, 0.3M, 0.4M, 0.5M, 0.6M, 0.7M, 0.8M, 0.9M, 1.0M, 1.1M, 1.2M, 1.3M, 1.4M, 1.5M, 1.6M, 1.7M, 1.8M, 1.9M, 2.0M, 2.1M, 2.2M, 2.3M, 2.4M, 2.5M, 2.6M, 2.7M, 2.8M, 2.9M, 3.0M, or 3.1M.

Some embodiments provide a Single Buffer (buffer II) that will extract at least 70% of the phytase predicted to be contained in an animal feed pellet compared to the amount included in the pre-pelleted mash. The phytase may retain phytase activity, and phytase may be quantified using a phytase activity assay, phytase quantification by ELISA, or both.

While aspects of the present invention have been described in some detail for purposes of clarity and understanding, one skilled in the art will appreciate that various changes in form and detail can be made without departing from the true scope of the invention.

### EXAMPLES

### Example 1

Raw Reagents and Stock Solutions: Sodium bicarbonate (S6014), Sodium deoxycholate (D6750), Triton X-100 (T9284), Tween 20 (P5927), Urea (U5128) were from Sigma. 1M CaCl2 (C0477), 1.0 M Tris pH 8.0 (T1080), 5.0 M NaCl (S0252), and 20× PBS pH 7.6 (P0191) were from Teknova. SDS (BP 166-500) was from Fisher. Guanidine (EMD 5010) was from EMD Chemicals. Chicken animal feed was property of Verenium Corporation.

Materials and instruments: Falcon 50 mL tubes (352096) were from BD Falcon; analytical balance (AT261) was from Mettler Toledo; centrifuge (5810R) was from Eppendorf; rotating wheel (099A RD4512) was from Glas Col; 25 mL pipettes (89130-900) were from VWR; Pipetman (22591) was from Thermo Scientific; and Vortex Genie-2 (12-812) was from Fisher.

### Example 2

Extraction Protocol 1 using High Stringency Buffer I(a), (b), and/or (c):

Adding a phytase to a mash, wherein the phytase has a known amount of phytase activity, mixing the mash, and subjecting the mash to a pelleting process.

A method for extracting a phytase from an animal feed comprising: providing an Animal feed (5 g) disposed in triplicates in 50 mL tubes. Adding a High Stringency buffer I(a), (b), and/or (c) (20 mL) to the animal feed, and vortex the composition for 5 seconds at maximum speed. Tubes were incubated on rotation wheel (60 rpm) for 1 h at room temperature. Tubes were spun down at 4,000 rpm in the centrifuge. Supernatant was separated from debris and kept at 4° C. for maximum 24 h.

High stringency buffer 1 was used to extract phytase enzymes from high-temperature treated animal feed pellets and the results are presented in Table 1, and FIG. 2.

TABLE 1
Percent of phytase mass extracted from a high temperature
pelleted animal feed relative to unpelleted precursor mash
comprising the phytase (quantified by ELISA).

| Buffer Name | 90° C. | 93° C. |
|---|---|---|
| High stringency buffer 1(a) Urea | 50.9% | 67.0 |
| | stdev = (+/−) 3.62% | stdev = (+/−) 4.9 |
| High stringency buffer 1(b) Guanidine | 81.5 | 101 |
| | stdev = (+/−) 4.0 | stdev = (+/−) 1.0 |
| High stringency buffer 1(c) mRIPA | 56.0 | 53.0 |
| | stdev = (+/−) 1.3 | stdev = (+/−) 1.0 |
| CAPS Buffer | 21.4 | N/A |
| (25 mM CAPS pH 10.0, 0.01% Tween20) | Stdev = (+/−) 6.2 | |
| PBST Buffer | 15.4 | N/A |
| (137 mM NaCl, 2.7 mM KCl, 10 mM Sodium | Stdev = (+/−) 5.0 | |
| Phosphate dibasic, 2 mM Potassium | | |
| Phosphate monobasic, pH 7.4, 0.01% | | |
| Twenn20) | | |

### Example 3

Extraction Protocol 1 and Single Buffer II:

Adding a phytase to a mash, wherein the phytase has a known amount of phytase activity, mixing the mash, and subjecting the mash to a pelleting process.

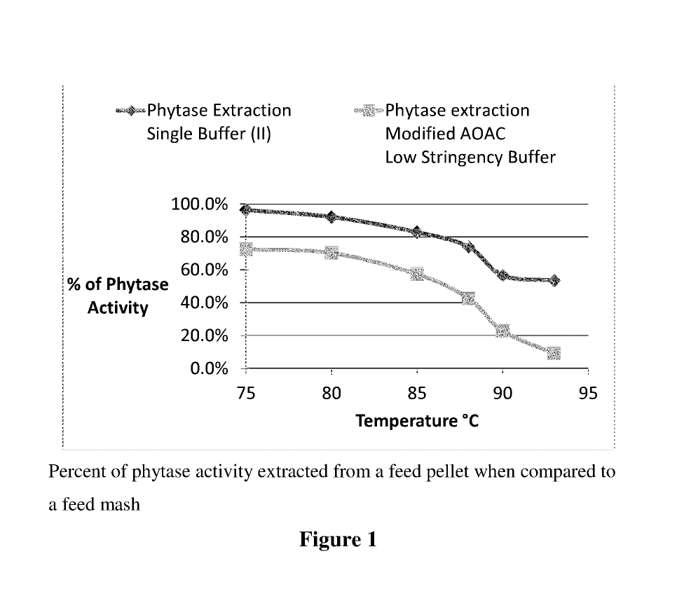

A method for extracting a phytase from an animal feed comprising: providing an animal feed (5 g) disposed in triplicates in 50 mL tubes; adding a single buffer (buffer II) (20 mL) to the animal feed and vortexed for 5 seconds at maximum speed; Tubes were incubated on rotation wheel (60 rpm) for 1 h at room temperature; Tubes were spun down at 4,000 rpm in the centrifuge; supernatant was separated from debris and kept at 4 C for maximum 24 h; and the phytase activity, the phytase quantity, and/or both were measured. Results were compared to results using an alternate buffer. See Table 2, and FIG. 1.

TABLE 2
Percent of phytase activity of phytase extracted from an animal
feed using various extraction buffers relative to the phytase
added to the mash prior to the pelleting process.

| | | | Difference in |
|---|---|---|---|
| | Extraction | Modified AOAC | percent extraction |
| | Protocol 1 | extraction | between Single |
| | Single Buffer II | Low Stringency Buffer | Buffer II and |
| Pelleting | Phytase | Phytase | Low Stringency |
| Temp. | Activity | Activity | Buffer |
| 75° C. | 96.4% | 72.5% | 23.9% |
| 80° C. | 92.2% | 70.3% | 21.9% |
| 85° C. | 82.9% | 57.4% | 25.5% |
| 88° C. | 74.0% | 42.6% | 31.4% |
| 90° C. | 56.5% | 22.9% | 33.6% |
| 93° C. | 53.6% | 9.2% | 44.4% |

### Example 4

Measuring Phytase Quantity by ELISA.

ELISA Buffers and proteins: ELISA Coating buffer: 1× PBS pH 7.6; ELISA Blocking Buffer: 1% BSA, 50 mM Tris pH 8.0, 100 mM NaCl, 0.01% Tween 20; Wash solution: 1× PBS pH 7.6, 0.02% Tween 20; Stop solution: 2 M sulfuric acid; phytase enzyme >90% purity; phytase antigen (expressed in Pichia pastoris) >90% purity.

Raw Reagents and Stock Solutions: BSA (A-7906), TMB solution (T0440) and Tween 20 (P5927) were from Sigma; 1M Tris pH 8.0 (T1080), 5.0 M NaCl (S0252) and 20× PBS pH 7.6 (P0191) were from Teknova; anti-biotin-HRP antibodies (GBIO-65P) were from Immunology Consultant Laboratories; sulfuric acid (A300-500) was from Fisher; anti-phytase antibodies (rabbit polyclonal, 4th bleed) were custom-made by ProSci using phytase; biotinylated anti-phytase antibodies were generated in house using anti-phytase antibodies; EZ-Link Sulfo-NHS-Biotin kit (21326) was from Thermo Scientific, used following manufacturer instructions.

Materials and instruments: 96-well plates (Costar 3590) were from Corning Inc.; Synergy H4 Hybrid Plate Reader and Plate Washer EL406 were from BioTek; multichannel pipettes were from Rainin.

Protocol for Phytase Quantification by ELISA: Anti-phytase capture antibodies (rabbit polyclonal, 4th bleed) were coated on a 96-well ELISA plate at 1 μg/mL concentration in coating buffer for 2 h at 23-25 C at 100 μL/well. Plate was washed three times with 300 μL wash solution using plate washer. Plate was blocked with 200 μL/well of blocking buffer for 1 h, at 23-25 C. Plate was washed three times with 300 μL wash solution using plate washer. Phytase containing samples were appropriately diluted into the ELISA blocking buffer (typically, ˜100-400 fold from pelleted feed extracts) and incubated for 1 h at 23-25 C at 100 μL/well. Similarly, phytase was diluted to 10-1500 pg/mL to generate standard curve. Plate was washed three times with 300 μL wash solution using plate washer. Biotinylated anti-phytase IgG (same with the capturing antibody) was diluted into blocking buffer at 0.4 μg/mL and incubated for 1 h at 23-25 C at 100 μL/well. Plate was washed three times with 300 μL wash solution using plate washer. Anti-biotin-HRP antibody was diluted to 0.2 μg/mL in ELISA blocking buffer and incubated for 1 h at 23-25 C at 100 μL/well. Plate was washed three times with 300 μL wash solution using plate washer. TMB solution (100 μL/well) was added and incubated for 10 min at 23-25 C. Reaction was stopped with 100 μL/well stop solution. Endpoint absorbance at 450 nm was recorded immediately by Synergy H4 Hybrid Plate Reader.

Definitions

As used herein, a bile salt detergent is a steroid acid compounded with a cation.

As used herein, a denaturant is a molecule that disrupts intermolecular or intramolecular protein interactions.

As used herein, “about” means plus or minus 20%.

As used herein, a chaotropic agent is an agent that disrupts boundaries in liquids separating aqueous and hydrophobic components. A chaotropic agent may also disrupt hydrophobic bonds involving a protein or proteins.

As used herein, a critical micelle concentration is a concentration of surfactants above which micelles form and all additional surfactants added to the system go to micelles.

As used herein, gentle agitation is sufficient to release enzymes from a heat-treated pellet.

As used herein, mash is a pellet precursor.

As used herein, amino acid residues are the side chains of amino acids that have been bound to form a polypeptide molecule, the sequence of said side chains specifying a protein.

As used herein, an enzyme is a protein catalyst.

The term “comprising” as used herein is synonymous with “including,” “containing,” or “characterized by,” and is inclusive or open-ended and does not exclude additional, un-recited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. An aqueous composition for extraction of a polypeptide from a heat-treated solid, said aqueous composition comprising a bile-salt detergent, a denaturant, a base, and water.

2. The composition of claim 1, wherein said denaturant is a chaotropic agent.

3.-6. (canceled)

7. The composition of claim 1, wherein said base is a bicarbonate salt.

8.-10. (canceled)

11. The composition of claim 1, wherein said aqueous composition has a basic pH.

12.-14. (canceled)

15. The composition of claim 1, wherein said bile-salt detergent comprises a steroid acid selected from the group consisting of taurocholic acid, glycocholic acid, cholic acid, deoxycholic acid, lithocholic acid, chenodeoxycholic acid, and any combination thereof.

16. The composition of claim 1, wherein said bile-salt detergent comprises a sodium cation.

17.-20. (canceled)

21. A method of extracting a polypeptide from a heat-treated feed pellet, comprising:
providing a heat-treated feed pellet comprising said polypeptide, said heat-treated pellet having been subjected to a heat treatment of at least 70° C.,
contacting said heat-treated feed pellet with an aqueous solution,
agitating said heat-treated feed pellet in contact with said aqueous solution to extract said polypeptide from said heat-treated feed pellet, and
removing said heat-treated feed pellet so as to obtain said extracted polypeptide in said aqueous solution.

22. The method of claim 21, wherein said aqueous solution comprises at least one detergent at a level of about or greater than a critical micelle concentration.

23. (canceled)

24. The method of claim 21, further comprising contacting said heat-treated feed pellet with a mild denaturant, wherein said mild denaturant disrupts intermolecular hydrophobic protein-protein interactions.

25. The method of claim 21, wherein said agitating comprises vortexing said heat-treated feed pellet in contact with said aqueous solution.

26. The method of claim 21 further comprising incubating said heat-treated feed pellet in contact with said aqueous solution.

27. (canceled)

28. The method of claim 26, wherein said incubating comprises a temperature of about 20° C. to 40° C., or a temperature of up to the denaturing temperature of said polypeptide.

29. The method of claim 26, wherein said polypeptide is an enzyme.

30.-34. (canceled)

35. A method for measuring activity of an enzyme in a heat-treated pellet comprising:
(a) providing a mash comprising an enzyme additive having a known enzyme activity;
(b) providing a heat-treated pellet generated from said mash;
(c) treating said mash and said heat-treated pellet with a low stringency buffer and measuring enzyme activity extracted through said treatment;
(d) treating said mash and said heat-treated pellet with a high stringency buffer and measuring the enzyme amount extracted through said treatment; and,
(e) determining an activity of the enzyme extracted from the heat-treated pellet.

36. The method of claim 35, wherein said low stringency buffer comprises water, about 50 mM Tris pH_{—}8.0, about 0.01% Tween 20, and about 10 mM CaCl₂.

37.-38. (canceled)

39. The method of claim 35, wherein said high-stringency buffer is selected from the group consisting of:
a Urea buffer comprising about 8.0 M Urea, about 50 mM Tris pH 8.0;
a Guanidine buffer comprising about 6.0 M Guanidine, about 50 mM Tris pH 8.0; and
a mRIPA buffer comprising about 50 mM Tris pH 7.6, about 0.15 M NaCl, about 0.1% SDS, about 0.5% sodium-deoxycholate, and about 1% Triton X-100.

40. The method of claim 35, wherein said high stringency buffer comprises 100 mM sodium bicarbonate pH10.0, 1.0% sodium deoxycholate and 1M urea.

41. The method of claim 35, wherein said enzyme additive is an animal feed enzyme additive.

42. The method of claim 35, wherein said known enzyme activity is a selected from the group consisting of phytase, cellulase, lactase, lipase, protease, catalase, xylanase, betaglucanase, mannanase, amylase, amidase, epoxide hydrolase, esterase, phospholipase, transaminase, amine oxidase, cellobiohydrolase, ammonia lyase, and any combination thereof.

43. The method of claim 35, wherein the amount of said enzyme is determined by ELISA.

44.-45. (canceled)
